(19) United States
(12) Patent Application Publication
(10) Pub. No.: US 2020/0022586 A1
(43) Pub. Date: Jan. 23, 2020

(54) WORKER MANAGEMENT APPARATUS, WORKER MANAGEMENT METHOD, AND RECORDING MEDIUM HAVING WORKER MANAGEMENT PROGRAM STORED THEREON

(71) Applicant: NEC Corporation, Tokyo (JP)

(72) Inventor: Shinya ESHIMA, Tokyo (JP)

(73) Assignee: NEC Corporation, Tokyo (JP)

(21) Appl. No.: 16/512,614

(22) Filed: Jul. 16, 2019

(30) Foreign Application Priority Data
Jul. 18, 2018 (JP) 2018-134876

(51) Int. Cl. A61B 5/0205; A61B 5/11; A61B 5/00

(52) U.S. Cl. CPC A61B 5/0205; A61B 5/681; A61B 5/1118

(57) ABSTRACT

A worker management apparatus includes: a work performance information generation unit generating work performance information representing a performance state of work by a worker; an acquisition unit acquiring physical information representing a physical condition relating to the worker; an estimation unit estimating an influence of the physical condition upon the performance state, based on the work performance information, the physical information, and information representing a characteristic of the worker; and an improvement measure generation unit generating improvement measure information representing a measure to improve the performance state relating to the worker, according to an estimation result by the estimation unit, thereby presenting a suitable measure to improve the work performance of the worker by managing with high precision the work performance state and physical condition relating to the worker.

This application is based upon and claims the benefit of priority from Japanese Patent Application No. 2018-134876, filed on Jul. 18, 2018, the disclosure of which is incorporated herein in its entirety by reference.

### TECHNICAL FIELD

The present invention relates to a technology for managing a work performance state and a physical condition relating to a worker.

### BACKGROUND ART

In recent years, much attention has been paid to work style reforms as one of challenges to be tackled by the administration, companies, or the like, against a backdrop of social problems such as a decrease in work performance efficiency due to work environment, living habit, and the like, and an increase in cost of health insurance. In promoting the work style reforms, expectations are rising for a technology for enabling implementation of appropriate improvement measures for improving work performance of a worker, by efficiently and suitably managing, with use of information technology (IT), a work performance state and a physical condition (health condition) relating to the worker.

As a related technology to the above technology, PTL 1 (International Publication No. WO 2010/146811) discloses an action proposing apparatus which estimates an action of a person, based on biological information such as sleeping hours, the number of steps, and an amount of exercise, and presents an improving means for improving the health index and the like. This apparatus classifies, as a habitual action, biological information (sensor data) representative of an identical feature in an identical time slot, from biological information and an action history (information) of a person acquired by a sensor node over a long period of time, and calculates, by an action guidance generation program, a correlation between time points, lengths of time, or a transitional relation of habitual actions, and target index data on which a user requests improvement. In addition, this apparatus displays major actions of the user in a time-series manner, and at the same time generates and presents on a display the optimal time and length of time for actions for improving the target index, and more specific examples of actions and comments for achieving the optimal time and length of time for the actions, by utilizing an action history which the user input and recorded in the past by himself/herself, and action history data input by other persons.

In addition, PTL 2 (Japanese Unexamined Patent Application Publication No. 2017-182393 A) discloses a biological information communication apparatus which enables easy sharing of biological information in a manner conforming with a user's intent. This apparatus receives biological information of a first user from the first user's user terminal being a transmission source, adds a restriction to the received biological information, and transmits the biological information, to which the restriction is added, to a user terminal of a second user, which is a transmission destination. This apparatus reads out, based on an acquired degree of correlation between the first user and the second user, restriction information that is associated with the degree of correlation and is stored in a restriction information storage unit, and adds a restriction to the biological information to be transmitted to the second user being a counter part of sharing, based on the read-out restriction information.

Additionally, PTL 3 (Japanese Unexamined Patent Application Publication No. 2017-108977 A) discloses a work support system which provides support information for facilitating work according to a health condition, and improves work efficiency of a worker. This system includes a fingertip pulse wave sensor which detects biological information. This system generates support information relating to work, based on the biological information, and provides the support information.

Additionally, PTL 4 (Japanese Unexamined Patent Application Publication No. 2014-191716 A) discloses a work management support system which supports management of a working state of a worker in such a way that the working state may be carried out as planned. This system supports management of the working state, based on working state data stored in a storage unit which stores working state data indicative of a working state of a worker being a target. This system detects working states of target workers on a worker-by-worker basis, based on working state data indicative of a working state of a target worker and working plan data indicative of a working plan of the target worker. Further, this system notifies a manager of a target worker of a message prompting a study of a work style reform of the target worker, in connection with whom a difference between the working plan of the target worker and the working state of the target worker exceeds a predetermined range.

Additionally, PTL 5 (Japanese Unexamined Patent Application Publication No. 2013-027550 A) discloses a vital signs measuring apparatus which detects vital signs and also detects necessary support information, and correctly reads vital signs while reducing a load on a subject and a doctor who conducts diagnosis. This apparatus detects vital signs of the subject, and outputs the vital signs as vital data. This apparatus detects support information representing a state of the subject at a time when the vital signs are detected, and outputs the support information as support data. Further, this apparatus performs evaluation as to whether the vital data is effective or not, based on the support data.

### SUMMARY

In order to make it possible to present a suitable measure to improve work performance of a worker by managing with high precision a physical condition based on physical information (biological information) of the worker and a work performance state of the worker, it is necessary, for example, to perform management by considering that physical characteristics and work performance ability vary from worker to worker. For example, there is a case in which, as regards two workers whose sleeping hours are equal, there is no particular problem in work performance efficiency of one of the workers, while work performance efficiency of the other worker lowers due to deficiency in sleep. In addition, for example, as regards creation of materials having similar contents, in the case where a certain worker requires three days for the creation while another worker requires four days for the creation, when work performance efficiency is evaluated, it is not possible to perform with high precision management as to whether the work performance efficiency of a worker lowers or not, unless consideration is given to such a difference in work performance ability. Further, when a work performance state and a physical condition relating to a worker fail to be managed with high precision, it is difficult to present a suitable measure to improve the work performances of the worker. PTLs 1 to 5 make no particular mention on such a problem, and it cannot be said that PTLs 1 to 5 are sufficient with respect to management with high precision of a work performance state and a physical condition relating to a worker. A main object of the present invention is to provide a worker management apparatus and the like which solve the problem.

### Solution to Problem

A worker management apparatus according to one mode of the present invention includes: a work performance information generation means for generating work performance information representing a performance state of work by a worker; an acquisition means for acquiring physical information representing a physical condition relating to the worker; an estimation means for estimating an influence of the physical condition upon the performance state, based on the work performance information, the physical information, and information representing a characteristic of the worker; and an improvement measure generation means for generating information representing a measure to improve the performance state relating to the worker, according to an estimation result by the estimation means.

In another aspect to achieve the above object, a worker management method according to one mode of the present invention includes causing an information processing apparatus to execute: generating work performance information representing a performance state of work by a worker; acquiring physical information representing a physical condition relating to the worker; estimating an influence of the physical condition upon the performance state, based on the work performance information, the physical information, and information representing a characteristic of the worker; and generating information representing a measure to improve the performance state relating to the worker, according to a result of the estimating.

In addition, in still another aspect to achieve the above object, a worker management program according to one mode of the present invention causes a computer to execute: generating work performance information representing a performance state of work by a worker; acquiring physical information representing a physical condition relating to the worker; estimating an influence of the physical condition upon the performance state, based on the work performance information, the physical information, and information representing a characteristic of the worker; and generating information representing a measure to improve the performance state relating to the worker, according to an estimation result by the estimation processing.

Further, the present invention can be achieved by a computer-readable nonvolatile recording medium having the worker management program (computer program) stored thereon.

### Advantageous Effects of Invention

The present invention makes it possible to present a suitable measure to improve a performance state of a worker, by managing with high precision a work performance state and a physical condition relating to the worker.

### BRIEF DESCRIPTION OF THE DRAWINGS

FIG. 1 is a block diagram which conceptually illustrates a configuration of a worker management system 1 according to a first example embodiment of the present invention;

FIG. 2 is s flowchart illustrating an operation of the worker management system 1 according to the first example embodiment of the present invention;

FIG. 3 is a block diagram which conceptually illustrates a configuration of a worker management apparatus 50 according to a second example embodiment of the present invention; and

FIG. 4 is a block diagram illustrating a configuration of an information processing apparatus 900 which can implement the worker management apparatuses according to the example embodiments of the present invention.

### EXAMPLE EMBODIMENT

Hereinafter, example embodiments of the present invention will be described in detail with reference to the accompanying drawings.

### First Example Embodiment

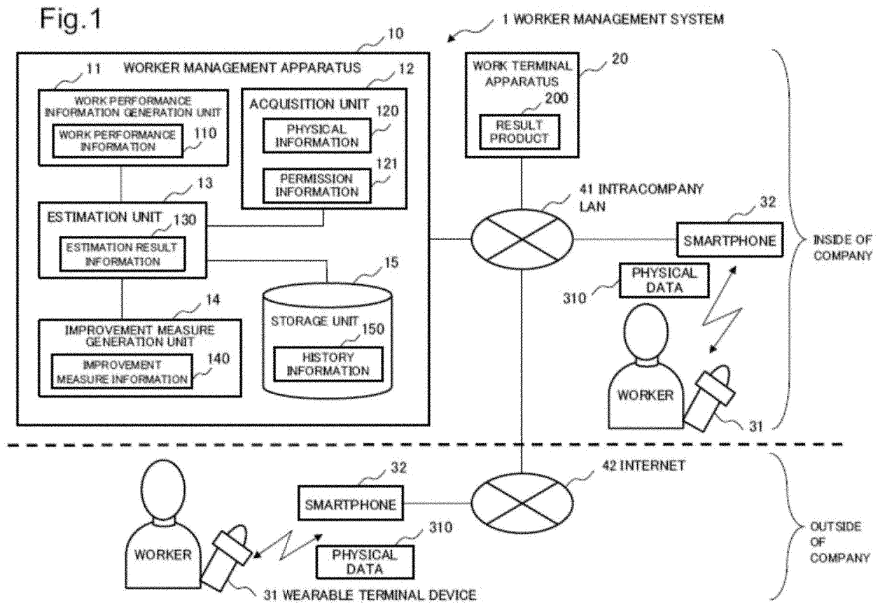

FIG. 1 is a block diagram which conceptually illustrates a configuration of a worker management system 1 according to a first example embodiment of the present invention. The worker management system 1 is a system which mainly includes a worker management apparatus 10, a work terminal apparatus 20, a wearable terminal device (measuring device) 31, and a smartphone 32, and in which these constituent elements are communicably connected via an intracompany local area network (LAN) 41 and Internet 42.

The wearable terminal device 31 is a device which can measure physical data 310 relating to at least one worker who is a management target of the worker management system 1, when necessary, regardless of working hours, the wearable terminal device 31 being, for example, wound around the arm of the worker, or being attached to the clothes of the worker. Note that the physical data 310 are, for example, data representing the physical condition of the worker, such as a heart rate, blood pressure, body temperature, and number of steps relating to the worker. The wearable terminal device 31 (or smartphone 32) may include, for example, a time-measuring function, and a function of acquiring position information utilizing a Global Navigation Satellite System (GNSS).

The wearable terminal device 31 and the smartphone 32 that the worker possesses can communicate by using a short-range communication function such as Bluetooth (trademark), and the wearable terminal device 31 transmits the measured physical data 310 to the smartphone 32. When the worker is outside the company (e.g. at home or on a commuting path), the smartphone 32 transmits the physical data 310, which is received from the wearable terminal device 31, to the worker management apparatus 10 through an access point (not illustrated) via the Internet 42 and intracompany LAN 41. When the worker is in the company, the smartphone 32 transmits the physical data 310, which is received from the wearable terminal device 31, to the worker management apparatus 10 through the access point via the intracompany LAN 41. Note that the wearable terminal device 31 may include a communication function which can transmit the physical data 310 to the worker management apparatus 10 without intervention of the smartphone 32.

The worker management apparatus 10 is an information processing apparatus which manages, with respect to at least one worker, the work performance state and physical condition of the worker, and can present, when there is a problem with the work performance state or physical condition of the worker, a measure to improve the work performance of the worker to the worker or a superior (manager) or the like of the worker.

The worker management apparatus 10 includes a work performance information generation unit 11, an acquisition unit 12, an estimation unit 13, an improvement measure generation unit 14, and a storage unit 15. The storage unit 15 is, for example, a storage device such as an electronic memory or a magnetic disk, which can store various pieces of information which the worker management apparatus 10 refers to or generates.

The work performance information generation unit 11 generates work performance information 110 representing the performance state of work (operation) by the worker. The work performance information generation unit 11 acquires a result product 200 from the work terminal apparatus 20, and generates the work performance information 110, based on the acquired result product 200. Note that the work terminal apparatus 20 is, for example, an information processing apparatus such as a personal computer, which the worker uses when doing work. The result product 200 is various materials to be considered, or the like, which the worker created as a result of the performance of work.

The work performance information 110 generated by the work performance information generation unit 11 is, for example, information representing the state of progress of creation of the result product 200 with reference to a creation schedule of the result product 200 (i.e. information indicating whether the creation of the result product 200 is earlier or later than the creation schedule). It is assumed, however, that the creation schedule is created depending on the work performance ability of the worker. The work performance information generation unit 11 can create the work performance information 110 by comparing a result of analyzing the content of the result product 200 by using an existing technology such as a syntax analysis technology, and information (not illustrated) representing the creation schedule.

Alternatively, the work performance information generation unit 11 may create the work performance information 110, based on information (not illustrated) representing a working state by the worker. Note that the information representing the working state by the worker is, for example, a report material representing the progress state of work, which was created by the worker himself/herself, or a management material which the superior of the worker created in order to manage the progress state of work by the worker. Alternatively, the information representing the working state by the worker may be, for example, a work quantity (e.g. the number of processed products) performed by the worker who performs work in a manufacturing line, the work quantity being acquired by an apparatus which can manage the work quantity.

Further, the work performance information generation unit 11 may generate the work performance information 110 representing the progress state of work per unit time (i.e. the efficiency of work), based on the progress state of work by the worker in a certain time slot.

The acquisition unit 12 acquires, based on permission information 121, the physical data 310 as physical information 120, the physical data 310 being measured by the wearable terminal device 31 and transmitted to the worker management apparatus 10. Note that the permission information 121 is information indicating which of the measured physical data 310 are permitted to be disclosed to the worker management apparatus 10, and is, for example, information created by the worker. Specifically, among the received physical data 310, the acquisition unit 12 acquires, as the physical information 120, only the data which are permitted to be disclosed by the permission information 121 by the worker.

The permission information 121 is, for example, information representing at least any one of items in the physical data 310, a time slot in which the physical data 310 were measured, and a range of values indicated by the physical data 310. For example, when the worker does not wish to disclose the heart rate, among the physical data 310 including the heart rate, blood pressure, body temperature and number of steps, the worker creates the permission information 121 indicating that the data of the items other than the heart rate are permitted to be disclosed to the worker management apparatus 10.

Alternatively, for example, when the worker does not wish to disclose the data measured in a specified time slot, among the physical data 310, the worker creates the permission information 121 indicating that the data measured in time slots excluding the specified time slot are permitted to be disclosed to the worker management apparatus 10. It is assumed, however, that the physical data 310 are associated with times at which the physical data 310 were measured, by the wearable terminal device 31 or smartphone 32, which includes a time-measuring function. Alternatively, the physical data 310 may be associated with times at which the worker management apparatus 10 received the physical data 310, by the worker management apparatus 10 which includes a time-measuring function.

Alternatively, for example, when the worker does not wish to disclose that the blood pressure, among the physical data 310, has become higher (or lower) than a specified value, the worker creates the permission information 121 indicating that the data relating to the blood pressure are permitted to be disclosed to the worker management apparatus 10 when the blood pressure is lower (or higher) than the specified value.

The permission information 121 may be stored in the storage unit 15 in the worker management apparatus 10, or may be stored in the wearable terminal device 31 or smartphone 32. For example, when the permission information 121 is stored in the smartphone 32, the smartphone 32 transmits to the worker management apparatus 10 only the data, the permission of disclosure of which is indicated by the permission information 121, among the physical data 310 received from the wearable terminal device 31.

The estimation unit 13 estimates an influence of the physical condition of the worker upon the work performance state, based on the work performance information 110 generated by the work performance information generation unit 11, the physical information 120 acquired by the acquisition unit 12, and the characteristics of the worker represented by history information 150 stored in the storage unit 15.

The history information 150 is information representing histories (states transitioning with the passing of time) of the work performance information 110 and physical information 120 relating to the worker, and is updated by the work performance information generation unit 11 and acquisition unit 12. Specifically, the history information 150 represents the relation between the performance state of work by the worker and the physical condition of the worker, with respect to a certain time slot in the past.

In addition, the estimation unit 13 estimates the action and condition of the worker, based on the history information 150 (the physical information 120 included in the history information 150). For example, when the history information 150 indicates that, in a certain time slot in the past, the worker stayed in the same position and the heart rate, blood pressure or the like meets a predetermined condition, the estimation unit 13 estimates that the worker was sleeping in the time slot.

Alternatively, for example, based on the heart rate, number of steps, speed of movement, etc., of the worker in a certain time slot in the past, which are indicated in the history information 150, the estimation unit 13 estimates that the worker performed exercise in the time slot. Alternatively, for example, based on the heart rate, body temperature, etc., of the worker in a certain time slot in the past, which are indicated in the history information 150, the estimation unit 13 estimates that the worker took a meal in the time slot. Alternatively, for example, when the history information 150 indicates that, in a certain period (e.g. several days) in the past, the body temperature of the worker was higher than an average value (e.g. normal temperature) relating to the worker, the estimation unit 13 estimates that the worker was sick in the period.

For example, based on the sleeping hours of the worker in a certain day, which is indicated in the history information 150 (the physical information 120 included in the history information 150), and the work efficiency of the worker in the next day, which is indicated in the history information 150 (the work performance information 110 included in the history information 150), the estimation unit 13 estimates an influence of the sleeping hours upon the work performance state of the next day with respect to the worker. For example, in the case where the history information 150 indicates that when the sleeping hours of the worker were less than six hours, the work efficiency of the worker of the next day tends to lower, the estimation unit 13 estimates that the worker has such characteristics that when the sleeping hours are less than six hours, the work efficiency of the next day lowers. Note that the estimation unit 13 can detect the tendency that the work efficiency lowers, based on the actual result of the work efficiency of the worker in the past, which is indicated by the history information 150. In addition, for example, the estimation unit 13 estimates that the work efficiency of the worker lowers due to the fact that the sleeping hours of the worker are less than six hours in recent successive days.

Alternatively, for example, in the case where the history information 150 indicates that when the worker did not take breakfast, the work efficiency of the worker in the day tends to lower, the estimation unit 13 estimates that the worker has such characteristics that when the worker does not take breakfast, the work efficiency of the worker in the day tends to lower. In addition, for example, the estimation unit 13 estimates that the work efficiency of the worker lowers due to the fact that the worker did not take breakfast in recent successive days.

Alternatively, for example, in the case where the history information 150 indicates that when the amount of exercise of the worker in a certain period meets a standard, the work efficiency of the worker in the period tends to improve, the estimation unit 13 estimates that the worker has such characteristics that when the worker takes suitable exercise, the work efficiency improves. In addition, for example, the estimation unit 13 estimates that the work efficiency of the worker lowers due to the fact that the worker has not recently taken suitable exercise.

Alternatively, for example, when the work efficiency of the worker has lowered, despite no problem being found in the daily life of the worker indicated in the history information 150, the estimation unit 13 estimates that the amount of work assigned to the worker is too large in consideration of the work performance ability of the worker.

As described above, the estimation unit 13 generates, as estimation result information 130, a result of estimation of the influence of the physical condition of the worker (the estimated action, etc. of the worker) upon the work performance state, based on the work performance information 110, the physical information 120, and the characteristics of the worker represented (estimated) by the history information 150.

The improvement measure generation unit 14 generates improvement measure information 140 representing a measure to improve the performance state relating to the worker, according to the estimation result information 130 generated by the estimation unit 13.

For example, when the estimation result information 130 indicates that the work efficiency of the worker lowers due to the fact that the sleeping hours of the worker are less than six hours in successive days, the improvement measure generation unit 14 generates the improvement measure information 140 which prompts the worker to make it sure to have six or more sleeping hours.

Alternatively, for example, when the estimation result information 130 indicates that the work efficiency of the worker lowers due to the fact that the worker did not take breakfast in recent successive days, the improvement measure generation unit 14 generates the improvement measure information 140 which prompts the worker to take breakfast.

Alternatively, for example, when the estimation result information 130 indicates that the work efficiency of the worker lowers due to the fact that the worker does not take suitable exercise, the improvement measure generation unit 14 generates the improvement measure information 140 which prompts the worker to take suitable exercise.

Alternatively, for example, when the estimation result information 130 indicates that the amount of work assigned to the worker is too large in consideration of the work performance ability, the improvement measure generation unit 14 generates the improvement measure information 140 representing information which prompts the superior of the worker to reduce the work load on the worker.

The improvement measure generation unit 14 may present the generated improvement measure information 140 to the worker or the superior of the worker, via the work terminal apparatus 20 or a terminal apparatus (not illustrated) which the superior of the worker uses.

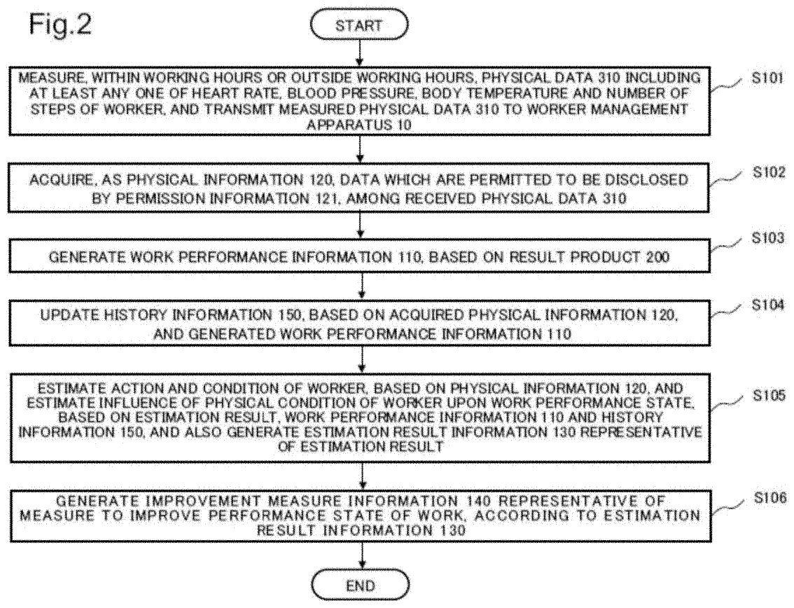

Next, referring to a flowchart of FIG. 2, a detailed description will be given of an operation (processing) of the worker management system 1 according to the present example embodiment.

The wearable terminal device 31 measures, within working hours or outside working hours, the physical data 310 including at least any one of the heart rate, blood pressure, body temperature and number of steps of the worker, and transmits the measured physical data 310 to the worker management apparatus 10, for example, via the smartphone 32 (step S101).

The acquisition unit 12 in the worker management apparatus 10 acquires, as the physical information 120, the data which are permitted to be disclosed by the permission information 121, among the received physical data 310 (step S102). The work performance information generation unit 11 generates the work performance information 110 by analyzing the result product 200 stored in the work terminal apparatus 20, for example, by using a syntax analysis technology or the like (step S103). Note that the processing of step S102 and the processing of step S103 are executed in no particular order, and may be executed, for example, in parallel.

The acquisition unit 12 updates the history information 150, based on the acquired physical information 120, and the work performance information generation unit 11 updates the history information 150, based on the generated work performance information 110 (step S104). The estimation unit 13 estimates the action and condition of the worker, based on the physical information 120, and estimates the influence of the physical condition of the worker upon the work performance state, based on this estimation result, the work performance information 110 and the history information 150, and also generates the estimation result information 130 representing this estimation result (step S105). The improvement measure generation unit 14 generates the improvement measure information 140 representing a measure to improve the performance state of the worker, according to the estimation result information 130 (step S106), and thus the entire processing ends.

The worker management apparatus 10 according to the present example embodiment can present a suitable measure to improve the work performance of the worker, by managing with high precision the work performance state and the physical condition relating to the worker. The reason for this is that the worker management apparatus 10 estimates the influence of the physical condition of the worker upon the work performance state, based on the work performance information 110 and physical information 120 relating to the worker, and the characteristics of the worker, and generates the improvement measure information 140 representing the measure to improve the work performance state relating to the worker, according to this estimation result.

Hereinafter, advantageous effects achieved by the worker management apparatus 10 according to the present example embodiment will be described in detail.

In order to make it possible to present a suitable measure to improve the work performance of a worker by managing with high precision the physical condition based on the physical information of the worker and the work performance state of the worker, it is necessary, for example, to perform the management by considering that the physical characteristics and work performance ability vary from worker to worker. For example, there is a case in which, as regards two workers whose sleeping hours are equal, there is no particular problem in the work performance efficiency of one of the workers, while the work performance efficiency of the other worker lowers due to the deficiency in sleep. In addition, for example, as regards the creation of materials having similar contents, in the case where a certain worker requires three days for the creation while another worker requires four days for the creation, when the work performance efficiency is evaluated, it is not possible to perform with high precision the management as to whether the work performance efficiency of workers lowers or not, unless consideration is given to such a difference in work performance ability. Further, when the work performance state and physical condition relating to workers fail to be managed with high precision, it is difficult to present suitable measures to improve the work performances of workers.

To cope with such problems, the worker management apparatus 10 according to the present example embodiment includes the work performance information generation unit 11, acquisition unit 12, estimation unit 13, and improvement measure generation unit 14, and operates as described above with reference to, for example, FIG. 1 and FIG. 2. Specifically, the work performance information generation unit 11 generates the work performance information 110 representing the performance state of work by the worker. The acquisition unit 12 acquires the physical information 120 representing the physical state relating to the worker. The estimation unit 13 estimates the influence of the physical condition of the worker upon the work performance state, based on the work performance information 110, the physical information 120, and information representing the characteristics of the worker. In addition, the improvement measure generation unit 14 generates the improvement measure information 140 representing the measure to improve the performance state of work relating to the worker, according to the estimation result information 130 generated by the estimation unit 13.

Specifically, the worker management apparatus 10 according to the present example embodiment can present a suitable measure to improve the performance state of a worker, by managing with high precision the work performance state and physical condition relating to the worker, taking into account the characteristics of the worker.

Additionally, the worker management apparatus 10 according to the present example embodiment stores in the storage unit 15 the history information 150 in which the history of the work performance information 110 and the history of the physical information 120 relating to the worker are associated, and estimates the influence of the physical condition of the worker upon the work performance state, by generating the information representing the characteristics of the worker, based on the history information 150. Thereby, since the worker management apparatus 10 according to the present example embodiment can estimate the characteristics of the worker with high precision, the worker management apparatus 10 can present a more suitable measure to improve the work performance of the worker.

Additionally, the estimation unit 13 according to the present example embodiment estimates the action or state (sleep, exercise, meal, etc.) of the worker, based on the physical information 120, and estimates, by using the estimation result, the influence of the physical condition of the worker upon the work performance state. Thereby, the worker management apparatus 10 according to the present example embodiment can enhance the precision when estimating the influence of the physical condition of the worker upon the work performance state.

Note that the estimation unit 13 according to the present example embodiment may use information different from the information based on the above-described history information 150, as the information representing the characteristics of the worker, which is used when estimating the influence of the physical condition of the worker upon the work performance state. Such information may be, for example, information in which a result of a questionnaire relating to the physical condition (including the condition of daily life, the state of felt stress, etc.), which was conducted to a worker, and the work performance state of the worker are associated.

Additionally, the worker management apparatus 10 according to the present example embodiment acquires, outside working hours (i.e. private time) as well as within working hours, the physical information 120 in order to manage with higher precision the physical condition of the worker. Accordingly, it is possible that the physical information 120 collected by the worker management apparatus 10 includes information which may greatly affect the worker in case of information leakage. Since the acquisition unit 12 according to the present example embodiment acquires the physical information 120 on the basis of the permission information 121 representing a permission by the worker to disclose the physical information 120 (i.e. the acquisition unit 12 does not acquire, as the physical information 120, the information, which the worker does not wish to disclose, among the measured physical data 310), the risk of the above-described information leakage can be avoided.

Additionally, the acquisition unit 12 according to the present example embodiment may acquire, as the physical information 120, for example, information representing the result of a health examination conducted on the worker, in addition to the physical data 310 measured by the wearable terminal device 31. In this case, since the worker management apparatus 10 according to the present example embodiment can estimate the influence of the physical condition of the worker upon the work performance state by taking into account the physical data which cannot be measured by the wearable terminal device 31, the worker management apparatus 10 can present a more suitable measure to improve the work performance of the worker.

Additionally, the work performance information generation unit 11 according to the present example embodiment generates the work performance information 110, based on the result product 200 which the worker created in connection with work, or the information representing the working state by the worker, or the like. Thereby, since the worker management apparatus 10 according to the present example embodiment can manage with high precision the work performance state of the worker, the worker management apparatus 10 can present a suitable measure to improve the work performance of the worker.

Additionally, the improvement measure generation unit 14 according to the present example embodiment may generate the improvement measure information 140 which also includes information or the like representing the result of an improvement of the work performance state of the worker, the result of the improvement being acquired by the worker implementing the improvement measure that was previously presented to the worker. Thereby, the worker management apparatus 10 according to the present example embodiment can help the superior of the worker to properly manage the worker.

### Second Example Embodiment

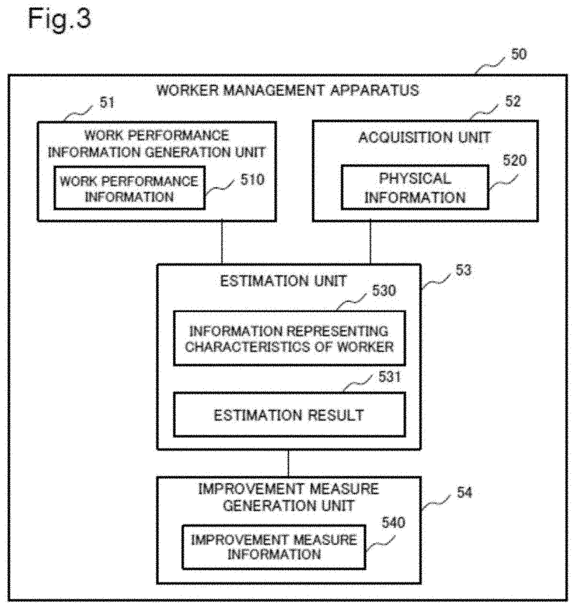

FIG. 3 is a block diagram which conceptually illustrates a configuration of a worker management apparatus 50 according to a second example embodiment of the present invention.

The worker management apparatus 50 according to the present example embodiment includes a work performance information generation unit 51, an acquisition unit 52, an estimation unit 53, and an improvement measure generation unit 54.

The work performance information generation unit 51 generates work performance information 510 representing the performance state of work by a worker.

The acquisition unit 52 acquires physical information 520 representing the physical condition relating to the worker.

The estimation unit 53 estimates an influence of the physical condition of the worker upon the work performance state, based on the work performance information 510, the physical information 520, and information 530 representing characteristics of the worker.

The improvement measure generation unit 54 generates improvement measure information 540 representing a measure to improve the performance state of work relating to the worker, according to an estimation result 531 by the estimation unit 53.

The worker management apparatus 50 according to the present example embodiment can present a suitable measure to improve the work performance of the worker, by managing with high precision the work performance state and physical condition relating to the worker. The reason for this is that the worker management apparatus 50 estimates the influence of the physical condition of the worker upon the work performance state, based on the work performance information 510 and physical information 520 relating to the worker, and the information 530 representing the characteristics of the worker, and generates the improvement measure information 540 representing the measure to improve the work performance state relating to the worker, according to the estimation result 531.

[Example of Hardware Configuration]

In each of the above-described example embodiments, the respective units in the worker management apparatuses illustrated in FIG. 1 and FIG. 3 can be achieved by exclusive HW (HardWare) (electronic circuitry). In addition, in FIG. 1 and FIG. 3, at least the configuration described below can be understood as function (processing) units (software modules) of a software program.
Work performance information generation units 11 and 51,
Acquisition units 12 and 52,
Estimation units 13 and 53, and
Improvement measure generation units 14 and 54.

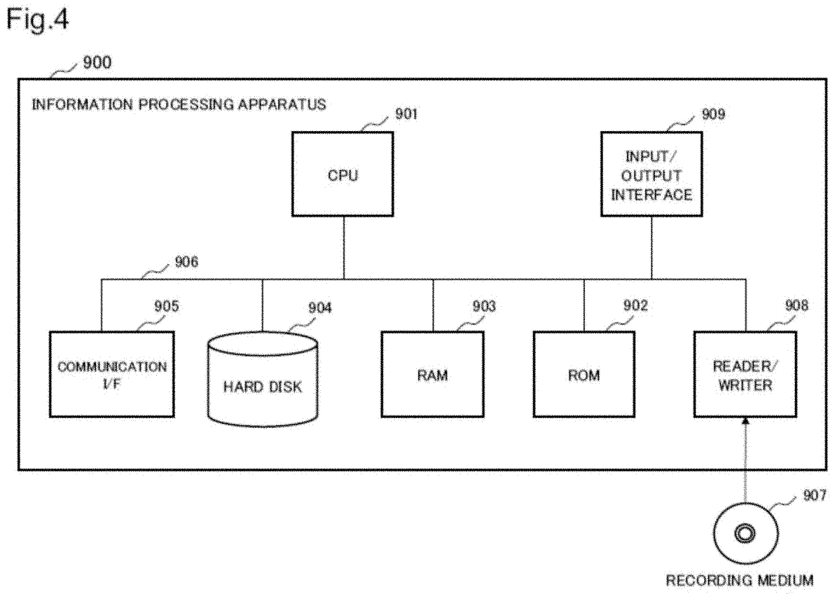

It should be noted, however, that the division of the units illustrated in the drawings is a configuration for the purpose of convenience of description, and various configuration can be assumed in implementation. An example of a hardware environment in this case will be described with reference to FIG. 4.

FIG. 4 is a view for illustratively describing a configuration of an information processing apparatus 900 (computer) which can implement the worker management apparatuses according to the example embodiments of the present invention. Specifically, FIG. 4 illustrates a hardware environment which is a configuration of a computer (information processing apparatus) that can implement the worker management apparatuses illustrated in FIG. 1 and FIG. 3, and which can implement the respective functions in the above-described example embodiments.

The information processing apparatus 900 illustrated in FIG. 4 includes the following as constituent elements:
CPU (Central_Processing_Unit) 901,
ROM (Read_Only_Memory) 902,
RAM (Random_Access_Memory) 903,
Hard disk (storage device) 904,
Communication interface 905,
Bus 906 (communication line),
Reader/writer 908 capable of reading/writing data stored in a recording medium 907 such as a CD-ROM (Compact_Disc_Read_Only_Memory), and
Input/output interface 909 such as a monitor, a speaker, a keyboard, etc.

Specifically, the information processing apparatus 900 including the above constituent elements is a general computer in which these constituent elements are connected via the bus 906. There is a case in which the information processing apparatus 900 includes a plurality of CPUs 901, and there is another case in which the information processing apparatus 900 includes the CPU 901 composed of multiple cores.

Further, the present invention described by taking the above-described example embodiments as examples provides a computer program which enables the information processing apparatus 900 illustrated in FIG. 4 to implement the following functions. The functions are the above-described configurations in the block configuration diagrams (FIG. 1 and FIG. 3) which were referred to in the description of the example embodiments, or the functions of the flowchart (FIG. 2). The present invention is then achieved by reading the computer program into the CPU 901 of the hardware, and interpreting and executing the computer program. Besides, the computer program which was supplied into the apparatus may be stored in a readable/writable volatile memory (RAM 903) or in a nonvolatile storage device such as the ROM 902 or hard disk 904.

Additionally, in the above case, a procedure, which is a general one at present, can be adopted as the method of supplying the computer program into the hardware. The procedure is, for example, a method of installing the computer program into the apparatus via the recording medium 907 of any kind such as a CD-ROM, or a method of downloading the computer program from the outside via a communication network such as the Internet. In such a case, the present invention can be understood as being composed by the code that constitutes the computer program, or by the recording medium 907 that stores the code.

While the invention has been particularly shown and described with reference to example embodiments thereof, the invention is not limited to these embodiments. It will be understood by those of ordinary skill in the art that various changes in form and details may be made therein without departing from the spirit and scope of the present invention as defined by the claims.

1. A worker management apparatus comprising:
a processor; and
a memory storing instructions to be executed by the processor by causing the processor to execute:
generating work performance information representing a performance state of work by a worker;
acquiring physical information representing a physical condition relating to the worker;
estimating an influence of the physical condition upon the performance state, based on the work performance information, the physical information, and information representing a characteristic of the worker; and
generating information representing a measure to improve the performance state relating to the worker, according to a result of the estimating.

2. The worker management apparatus according to claim 1, wherein
the processor acquires the physical information, based on permission information representing a permission by the worker to disclose the physical information.

3. The worker management apparatus according to claim 2, wherein
the permission information represents at least any one of an item in the physical information, a time slot in which the physical information is measured, and a range of a value indicated by the physical information.

4. The worker management apparatus according to claim 1, wherein
the processor generates the work performance information, based on a result product which the worker creates in connection with the work, or information representing a working state by the worker.

5. The worker management apparatus according to claim 1, wherein
the processor estimates an action or a state of the worker, based on the physical information.

6. The worker management apparatus according to claim 1, wherein
the memory stores history information in which a history of the work performance information and a history of the physical information relating to the worker are associated with each other, and
the processor generates the information representing the characteristic of the worker, based on the history information.

7. The worker management apparatus according to claim 1, further comprising
a measuring device configured to measure data included in the physical information, and to transmit the measured data to the processor.

8. The worker management apparatus according to claim 7, wherein
the measuring device is configured to measure, as the data, at least any one of a heart rate, a blood pressure, a body temperature, and a number of steps relating to the worker.

9. A worker management method comprising
causing an information processing apparatus to execute:
generating work performance information representing a performance state of work by a worker;
acquiring physical information representing a physical condition relating to the worker;
estimating an influence of the physical condition upon the performance state, based on the work performance information, the physical information, and information representing a characteristic of the worker; and
generating information representing a measure to improve the performance state relating to the worker, according to a result of the estimating.

10. A recording medium having a worker management program stored thereon, the program causing a computer to execute:
generating work performance information representing a performance state of work by a worker;
acquiring physical information representing a physical condition relating to the worker;
estimating an influence of the physical condition upon the performance state, based on the work performance information, the physical information, and information representing a characteristic of the worker; and
generating information representing a measure to improve the performance state relating to the worker, according to an estimation result by the estimation processing.
